(19) United States
(12) Patent Application Publication
(10) Pub. No.: US 2018/0021006 A1
(43) Pub. Date: Jan. 25, 2018

(54) RADIOGRAPHIC APPARATUS, RADIOGRAPHIC SYSTEM, RADIOGRAPHIC METHOD, AND STORAGE MEDIUM

(71) Applicant: CANON KABUSHIKI KAISHA, Tokyo (JP)

(72) Inventor: Toru Takasawa, Kawasaki-shi (JP)

(21) Appl. No.: 15/649,490

(22) Filed: Jul. 13, 2017

(30) Foreign Application Priority Data
Jul. 22, 2016 (JP) 2016-144772

(51) Int. Cl. A61B 6/00

(52) U.S. Cl. CPC A61B 6/548; A61B 6/566; A61B 6/545; A61B 6/461

(57) ABSTRACT

A radiographic apparatus includes an enabling unit provided in a first radiation detector that enables the first radiation detector, a setting unit provided in the first radiation detector that sets image capturing information of the first radiation detector, and a control unit that, in a case where the first radiation detector is enabled by the enabling unit, controls the first radiation detector and a second radiation detector based on the image capturing information set by the setting unit.

### BACKGROUND

### Field of the Invention

The present disclosure relates to a radiographic apparatus, a radiographic system, a radiographic method, and a storage medium.

### Description of the Related Art

Conventionally, there is a radiographic image capturing system using a radiographic apparatus for causing a radiation generating apparatus to irradiate a patient's body with radiation, digitalizing radiation data, which is the intensity distribution of radiation passing through the patient's body, and performing image processing on a digitalized radiographic image, thereby generating a vivid radiographic image.

Such a radiographic image capturing system includes a radiation generating apparatus, a radiation detector, and a control computer for controlling the state of the radiation generating apparatus, or performing image processing on a captured image transferred from the radiation detector, or transmitting and receiving various types of data to and from an external information system. Radiographic image data generated by a radiographic apparatus as the result of the radiation generating apparatus emitting radiation is transferred to the control computer so that the radiographic image data is subjected to image processing or saved. Then, a processed image is displayed on a display apparatus.

As the radiation detector, a flat panel detector (FPD) is known in which solid-state image sensors are placed in a two-dimensional matrix. The FPD includes a photoelectric conversion circuit in which a plurality of photoelectric conversion elements for converting radiation into electric signals is arranged in a matrix, and a reading circuit for reading from the photoelectric conversion circuit the electric signals obtained by this conversion.

Radiation passing through a patient's body is converted by a fluorescent body into light based on the amount of radiation. The converted light is photoelectrically converted by the photoelectric conversion elements of the photoelectric conversion circuit. Signal charges corresponding to the amounts of transmitted radiation are accumulated in the respective photoelectric conversion elements. The reading circuit drives signal lines of the photoelectric conversion circuit and appropriately controls switch elements to which the photoelectric conversion elements are connected. In this way, the signal charges accumulated in the respective photoelectric conversion elements are sequentially read as electric signals, which are then amplified to be output as amplified electric signals.

The FPD irradiated with radiation includes a state where the FPD can acquire data (an enabled state) and a state where the FPD cannot acquire data (a disabled state). If the FPD in the enabled state receives a synchronization signal from an emission switch, the FPD starts a reading operation.

In recent years, an FPD has been introduced that acquires a transmitted radiographic image by automatically detecting radiation without synchronizing with the emission timing of a radiation generating apparatus. This type of radiation detector does not require a cable for synchronization, and therefore, is advantageous in that the radiation detector can be combined with any radiation generating apparatus.

A configuration has also been proposed in which a memory for storing an acquired image is provided in an FPD, thereby eliminating the need for a control computer when an image is captured. In this configuration, after an examination, the FPD in which a captured image is saved is connected to the control computer, a radiographic image is transferred, and image processing is performed on the radiographic image. Then, the resulting image is associated with patient information and part information.

The above-described radiation detector is, as a cassette-type, available in various sizes, such as large square (35 cm×35 cm), full (43 cm×43 cm), and large quarter (27 cm×35 cm). The radiation detector can be installed in a stand or a bed, and an image is captured by various image capturing techniques. For example, to capture a patient's fingers, a large-quarter-size radiation detector is used. To capture a patient's abdomen by placing the radiation detector under the patient's bed, a full-size radiation detector is used.

With a radiation detector installed in a stand, a patient's front chest can be captured. With a radiation detector installed in a bed, in addition to capturing a patient's abdomen, as previously described, a patient's joints can be captured. In a case where a patient cannot be captured in a standing position because, for example, the patient's legs are weak, a stand-type radiation detector is changed to a bed-type radiation detector based on the condition of the patient when an image is captured. At this time, the radiation detectors are switched via an operation unit connected to the control computer. As described above, depending on the image capturing technique or the build and the state of the patient, the radiation detectors are switched for use. Generally, however, an operation console for controlling X-rays and an operation unit for operating radiation detectors are located in an operation room different from an imaging room where an image is captured.

A radiologic technologist, as an operator, switches to a radiation detector suitable for an image capturing technique using the operation unit located in the operation room, returns to the imaging room, and positions a patient relative to the selected radiation detector. At this time, for example, in a case where the size of the radiation detector is different or the image capturing technique is different in addition to the size of the radiation detector, the radiologic technologist needs to return to the operation room again and perform an operation using the operation unit.

Japanese Patent Application Laid-Open No. 2015-6413 discusses a technique in which a selection button is provided on a radiation detector and the selected radiation detector is associated with patient information for a case where a plurality of radiation detectors is switched. Japanese Patent No. 3890163 discusses a technique in which a button provided corresponding to each radiation detector is pressed to bring the radiation detector and a phototimer into a normal current state (an enabled state) and control a non-selected radiation detector and a phototimer to enter a low current state (a disabled state).

The above referenced cases do not discuss a case where image capturing techniques are changed together with radiation detectors. Thus, it is necessary to set image capturing information regarding an image capturing technique after an examination. This results in poor operability.

### SUMMARY

According to an aspect of the present invention, a radiographic apparatus includes an enabling unit provided in a first radiation detector and configured to enable the first radiation detector, a setting unit provided in the first radiation detector and configured to set image capturing information of the first radiation detector, and a control unit configured to, in a case where the first radiation detector is enabled by the enabling unit, control the first radiation detector and a second radiation detector based on the image capturing information set by the setting unit.

Further features will become apparent from the following description of exemplary embodiments with reference to the attached drawings.

### BRIEF DESCRIPTION OF THE DRAWINGS

FIG. 1 is a block diagram illustrating a general configuration of a radiographic image capturing system according to a first exemplary embodiment.

FIG. 2 is a diagram illustrating an example of an image capturing protocol selection unit.

FIG. 3 is a diagram illustrating an example of an image capturing protocol list.

FIG. 4 is a diagram illustrating an example of an image capturing preparation screen.

FIG. 5 is a diagram illustrating an example of an image capturing screen.

FIG. 6 is a diagram illustrating an example of a detector switching window.

FIG. 7 is a block diagram illustrating a detailed configuration of the radiographic image capturing system according to the first exemplary embodiment.

FIG. 8 is a flowchart illustrating an enabling/disabling process in the first exemplary embodiment.

FIG. 9 is a flowchart illustrating processing after an image capturing protocol is input in the first exemplary embodiment.

FIG. 10 is a diagram illustrating examples of input units for inputting a part, an age category, an image capturing direction, and rotation information.

FIG. 11A is a diagram illustrating an example of a list of parts, FIG. 11B is a diagram illustrating an example of a list of age categories, and FIG. 11C is a diagram illustrating an example of a list of image capturing directions.

FIG. 12 is a diagram illustrating an example of a database of a protocol identification (ID), a part, an age category, and an image capturing direction.

FIG. 13 is a diagram illustrating an example of a direction specifying unit.

FIG. 14 is a block diagram illustrating a detailed configuration of a radiographic image capturing system according to a second exemplary embodiment.

FIG. 15 is a block diagram illustrating a general configuration of a radiographic image capturing system according to a third exemplary embodiment.

### DESCRIPTION OF THE EMBODIMENTS

Exemplary embodiments of a radiographic image capturing system will be described below with reference to the drawings. The following examples illustrated in the figures are not seen to be limiting.

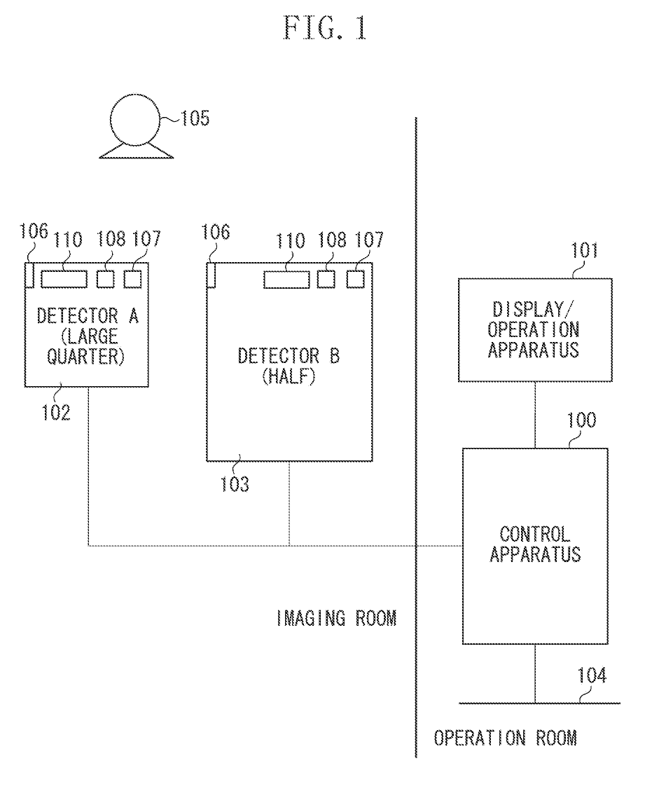

FIG. 1 illustrates a general configuration of a radiographic image capturing system according to a first exemplary embodiment. FIG. 1 illustrates a detector A (first radiation detector) 102 and a detector B (second radiation detector) 103. Both detector A 102 and detector B 103 detect radiation passing through a patient's body and output the distribution of the transmitted radiation as image data. Detector A 102 is 27 cm wide and 35 cm high, and detector B 103 is 43 cm wide and 43 cm high. A patient's fingers and four limbs have narrow image capturing areas, and are thus captured using detector A 102. A patient's chest and abdomen have relatively wide image capturing areas, and are thus captured using detector B 103.

A control apparatus 100 controls detector A 102 or detector B 103 and a display/operation apparatus 101, and communicates patient information, examination information, implementation information, and image data to and from an external information system via a hospital network 104. According to the state of the system, the control apparatus 100 causes detector A 102 or detector B 103 to transition to an enabled state or a disabled state. The control apparatus 100 also performs various correction processing and various types of image processing on transferred image data to generate a captured image, and displays the captured image on the display/operation apparatus 101.

The “enabled state” refers to the state where a reading circuit of both detectors A 102 and B 103 is driven, and image data can be acquired in response to irradiation. The “disabled state” refers to the state where image data cannot be acquired, even if detectors A 102 and B 103 are being irradiated, such as the state where power is not supplied to the reading circuit or a low current state. In the case of an automatic detection type, the state where irradiation cannot be detected corresponds to the disabled state.

An enabling button 107 and a disabling button 108 are used to cause detector A 102 or detector B 103 to transition to the enabled state and the disabled state, respectively. In the enabled state, a “ready” lamp 106 lights up in green. The enabling button (enabling unit) 107 for enabling detector A (first radiation detector) 102 is included in detector A 102. For example, the enabling button (enabling unit) 107 is provided in a housing or a support for accommodating detector A (first radiation detector) 102 for detecting radiation and enables detector A (first radiation detector) 102.

An image capturing protocol identification (ID) input unit (setting unit) 110 for setting image capturing information of detector A (first radiation detector) 102 is provided in detector A 102. For example, the image capturing protocol ID input unit (setting unit) 110 is provided in a housing or a support for accommodating detector A (first radiation detector) 102 and sets image capturing information of detector A (first radiation detector) 102. The image capturing information includes at least one of an image capturing protocol ID, a captured part, an image capturing direction, an age category of a subject, or rotation information of a radiographic image.

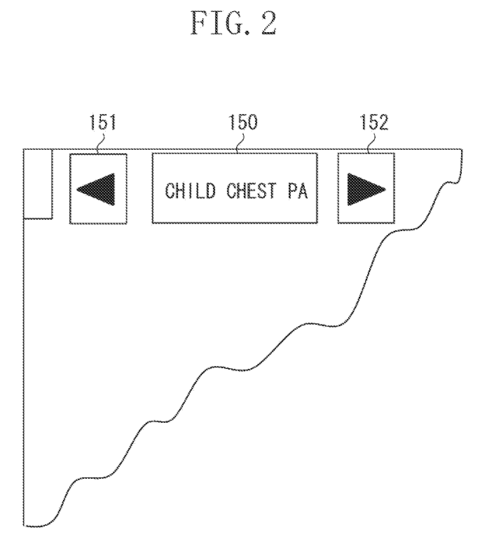

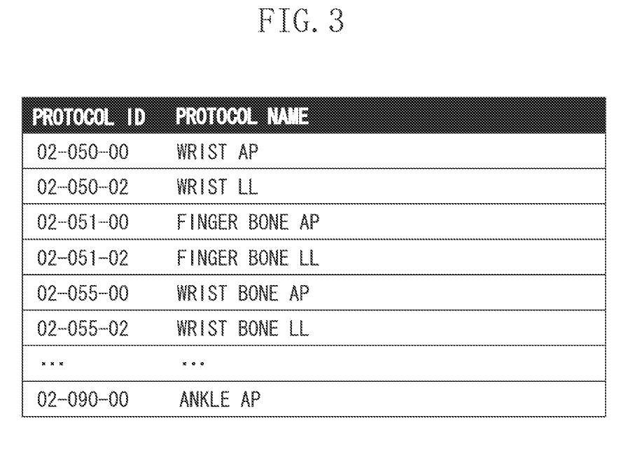

For example, the image capturing protocol ID input unit (setting unit) 110 is used to input an image capturing protocol ID. As illustrated in FIG. 2, the image capturing protocol ID input unit 110 includes an image capturing protocol display area 150 and change buttons 151 and 152. FIG. 3 illustrates a protocol ID list of the protocol IDs of protocols with which an image can be captured by an X-ray detector. Based on this list, the name of a selected protocol is displayed in the image capturing protocol display area 150. Both change buttons 151 and 152 are buttons used to change and display image capturing protocol IDs in the image capturing protocol display area 150 in descending or ascending order.

For the image capturing protocol ID input unit 110, a method of directly inputting a protocol ID can be used. In this case, numeric keypad input, sound input, or input from external information input source, such as a magnetic card or a barcode, are also possible.

A radiation source 105 generates radiation and, for example, corresponds to an X-ray tube. The radiation source 105 is part of a radiation generating apparatus (not illustrated) and an operation console of the radiation generating apparatus. A radiologic technologist inputs imaging conditions using the operation console and presses an emission switch, thereby emitting radiation from the radiation source 105. The radiation generating apparatus is connected to the network 104 so that the display/operation apparatus 101 sends imaging conditions, such as a tube voltage, a tube current, and an emission time, to the radiation generating apparatus to set the imaging conditions. The radiation generating apparatus can also send the imaging conditions, under which an image is captured, to the display/operation apparatus 101.

The display/operation apparatus 101 displays an image and enables operation of the image capturing system. A touch panel monitor is typically used as the display/operation apparatus 101. Alternatively, the display/operation apparatus 101 can be combined with input devices such as a mouse, a keyboard, a magnetic card, or a barcode reader.

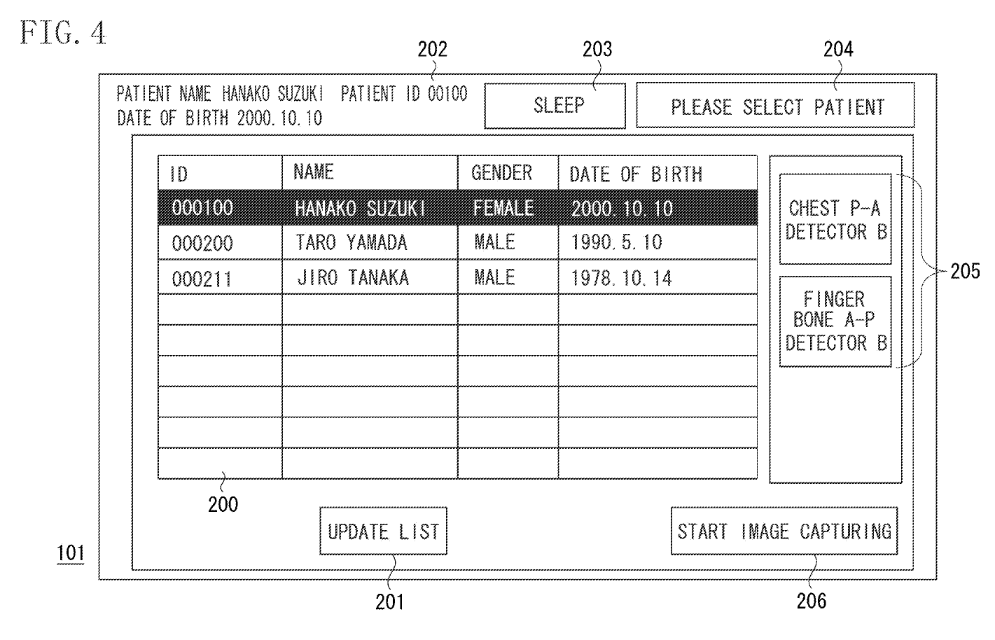

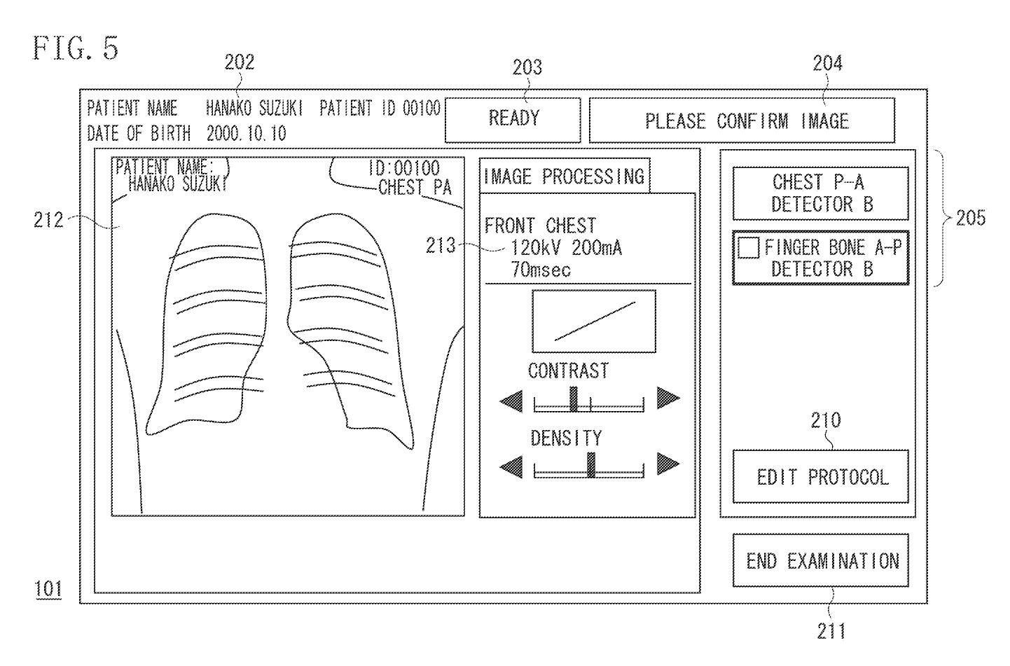

FIGS. 4 and 5 illustrate examples of graphical user interface (GUI) screens displayed on the display/operation apparatus 101. FIG. 4 illustrates an image capturing preparation screen on which an image capturing order list is displayed. FIG. 5 illustrates an image capturing screen after an image is captured.

FIG. 4 illustrates the state where patient information and examination information sent from a radiology information system (RIS) via the network 104 are displayed as an image capturing order list 200. The radiologic technologist selects a corresponding patient from the image capturing order list 200, confirms the selected patient in a patient information display area 202 and an image capturing protocol display area 205, and presses a “start image capturing” button 206.

In the image capturing protocol display area 205, protocols are arranged in the order of image capturing and executed in order from the top. Each image capturing protocol is configured by the combination of a part to be captured, an image capturing direction, and a radiation detector to be used. If an image capturing protocol is specified, these items are uniquely determined. Image processing parameters to be used and calibration data for each radiation detector are also determined. An “update list” button 201 is a button for updating the image capturing order list 200.

FIG. 5 illustrates the state where a captured image obtained by irradiating a patient's body with X-rays is displayed. In an image display area 212, a captured image is displayed, and various types of image processing in an image processing toolbox 213 are adjusted, thereby adjusting the contrast, the density, and the sharpness of the image.

The image capturing protocol display area 205 means in the upper part that the capturing of an image using “chest P-A” has ended, and the image is displayed in the image display area 212. The image capturing protocol display area 205 also means in the lower part that the focus is placed on “finger bone A-P”, and an image will be captured from now.

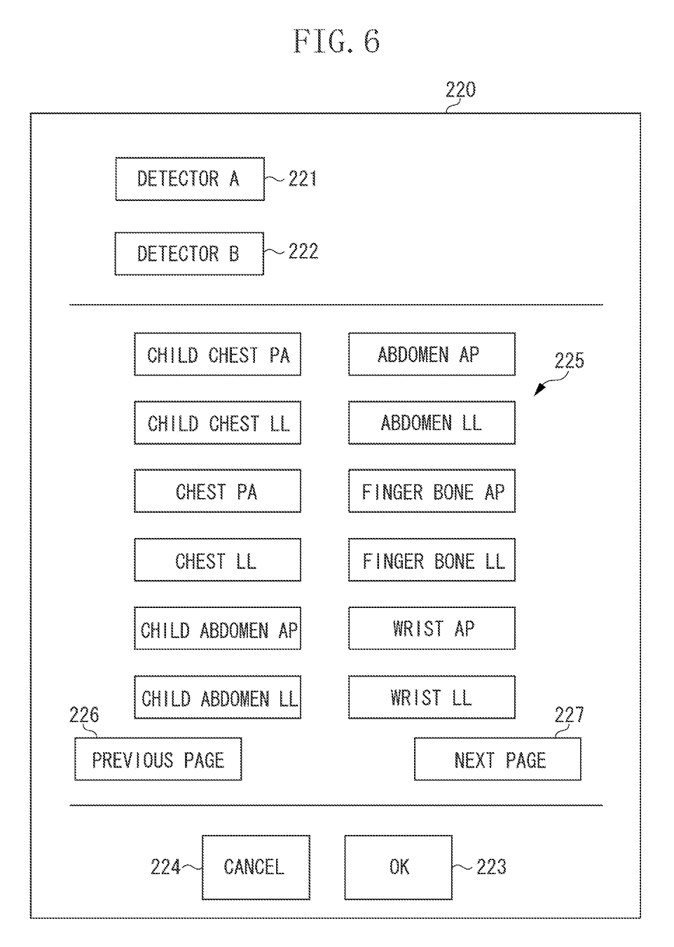

The radiation detectors 102 and 103 of the protocol on which the focus is placed can be changed using an “edit image capturing protocol” button 210. If the “edit image capturing protocol” button 210 is pressed, an image capturing protocol change window 220 in FIG. 6 is displayed, and the radiation detectors 102 and 103 and the image capturing protocols can be switched. Image capturing protocols corresponding to either of the radiation detectors 102 and 103 selected using detector selection buttons 221 and 222 are displayed in an image capturing protocol selection area 225. Although an image capturing protocol is selected here, displayed pages can be switched using page switching buttons 226 and 227.

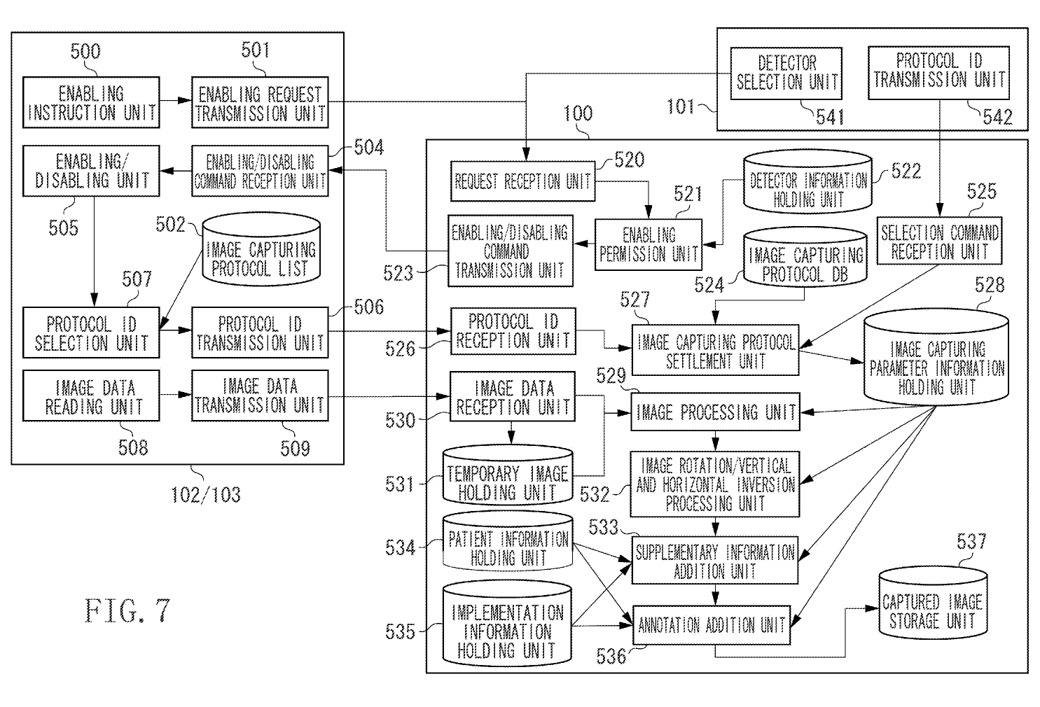

FIG. 7 is a block diagram illustrating an example of the radiographic system according to the present exemplary embodiment.

First, if the radiologic technologist selects a patient on the image capturing preparation screen in FIG. 4 and presses the “start image capturing” button 206, the “chest P-A” protocol, which is the first protocol, is automatically selected, and the image capturing preparation screen transitions to the image capturing screen. Then, the detector B 103 is enabled, and the “ready” lamp 106 of the detector B 103 lights up. At this time, the patient information is stored in a memory 534 for storing patient information.

The radiologic technologist positions the patient so that the relative position between the detector B 103 and the patient is appropriate in an imaging room. Then, the radiologic technologist goes to an operation room, confirms, using the operation console of the radiation generating apparatus, whether imaging conditions are correct, and presses the emission switch to emit radiation.

Radiation passing through the patient's body is detected by the detector B 103 and sent as image data to the control apparatus 100. The control apparatus 100 performs correction processing using the calibration data corresponding to the detector B 103, and then performs various types of image processing such as contrast processing, density processing, sharpness processing, and dynamic compression processing to generate a captured image. The generated captured image is sent to the display/operation apparatus 101 and displayed in the image display area 212 of the image capturing screen in FIG. 5.

Then, the focus shifts to “finger bone A-P”, which is the next image capturing protocol, and a next image can be captured. In response, the technologist returns to the imaging room and positions the patient to capture the patient's fingers using the detector B 103 in the enabled state. It is, however, understood that the image capturing area of the fingers is narrow, and therefore, a size of 43 cm×43 cm is not necessary. Thus, to capture an image by switching to the detector A 102 of a large quarter size, which is located in the same imaging room, the radiologic technologist presses the enabling button 107 attached to the detector A 102.

The enabling button 107 is one of various methods illustrated as an enabling instruction unit 500 in FIG. 7. Instead of a button, a switch or a sound input device can be used as long as an instruction to enable the detector A 102 can be provided.

If input is received from the enabling instruction unit 500, an enabling request transmission unit 501 transmits an enabling request to the control apparatus 100. The transmitted enabling request is received by a request reception unit 520, and an enabling permission unit 521 determines whether the detector A 102 can be enabled. The enabling request command also includes information of the ID of the detector A 102 as the transmission source. After the state of the radiation detector is confirmed based on detector information in a detector information holding unit 522, and if there is no contradiction, the enabling permission unit 521 permits an enabling process.

If the enabling process is permitted, the enabling permission unit 521 performs an exclusive process using, for example, a semaphore and mutex in a program to prevent another state transition request from interrupting. Based on the detector information in the detector information holding unit 522, an enabling/disabling command transmission unit 523 issues a disabling command to the detector B 103 in the enabled state. The issued disabling command is received by an enabling/disabling command reception unit 504 of the detector B 103 and sent to an enabling/disabling unit 505. The detector B 103 transitions to the disabled state, and the “ready” lamp 106 goes out.

Next, the enabling/disabling command transmission unit 523 issues an enabling command to the detector A 102, and the control apparatus 100 updates the detector information in the detector information holding unit 522. In the detector A 102, an enabling/disabling unit 505 receives the enabling command from an enabling/disabling command reception unit 504 and causes the detector A 102 to transition to the enabled state.

In the enabled state, the “ready” lamp 106 lights up. The control apparatus 100 updates the detector information in the detector information holding unit 522. If the detector A 102 is enabled by the enabling/disabling unit 505, a protocol ID selection unit 507 is enabled so that a protocol ID can be selected.

As described above, the detector A 102 enters the enabled state, and the patient's fingers can be captured using the detector A 102. If the radiologic technologist views the state of the patient and finds that it is necessary to capture the patient's wrist instead of the fingers, the radiologic technologist presses the image capturing protocol change buttons 151 and 152, which correspond to the enabled protocol ID selection unit 507, to select “wrist AP”, and settles “wrist AP” using a settlement button (not illustrated).

At this time, every time each of the change buttons 151 and 152 is pressed, the image capturing protocol list 502 (FIG. 3) is displayed in ascending order or descending order. In the image capturing protocol list 502, appropriate image capturing methods are listed for each radiation detector. At this time, when the control apparatus 100 issues the enabling command from the enabling/disabling command transmission unit 523 to the detector A 102, the protocol ID of “finger bone A-P”, which is a selected image capturing protocol, can also be transmitted together with the enabling command.

In this case, the display of the image capturing protocol display area 150 can be changed to “finger bone A-P”, which is selected by the control apparatus 100.

The control apparatus 100 can analyze the capturing of images in the past in the radiographic image capturing system and list protocols so that the protocols are displayed in the order of image capturing frequency in the image capturing protocol display area 150. As described above, the image capturing protocol ID input unit (setting unit) 110 can set image capturing information of the detector A 102 from image capturing information ordered based on priority based on past image capturing information of the detector A (first radiation detector) 102. The image capturing protocol ID input unit 110 can set image capturing information of the detector A 102 from image capturing information ordered based on priority based on past image capturing information of the detector B 103.

Alternatively, the image capturing protocol ID input unit (setting unit) 110 can set image capturing information of the detector A 102 from image capturing information ordered based on priority based on past image capturing information of the detector A 102 and the detector B 103. In this case, as will be described below, the detector A 102 and the detector B 103 can share the image capturing information ordered based on priority based on the past image capturing information of the detector A 102 and the detector B 103.

Specifically, as the result of analyzing the capturing of images in the past in the radiographic image capturing system, if protocols most frequently implemented, in descending order, are “wrist A-P”, “wrist L-L”, and “finger bone A-P” according to the order of image capturing frequency, the protocols are displayed in the order of “wrist A-P”, “wrist L-L”, and “finger bone A-P” in the image capturing protocol display area 150. In the image capturing protocol display area 150 in an initial state, “wrist A-P”, which is most frequently used to capture images, is displayed. As described above, protocols are displayed in the order of image capturing frequency (the order of setting frequency) in the image capturing protocol display area 150. Thus, the burden of selecting a protocol is reduced.

As described above, the priority of image capturing information can be determined based on the setting image capturing frequency information, or can be determined based on the setting time, for example, such that protocols are displayed in the reverse chronological order of the setting time in the image capturing protocol display area 150.

The control apparatus 100 can list protocols that can be commonly used by the radiation detectors 102 and 103, so that the protocols are displayed in the image capturing protocol display area 150. Specifically, if “chest P-A”, “chest A-P”, and “ankle joint A-P” protocols can be commonly used by the radiation detectors 102 and 103, “chest P-A”, “chest A-P”, and “ankle joint A-P” are displayed in order in the image capturing protocol display area 150. Since protocols that can be commonly used by the radiation detectors 102 and 103 are displayed in order in the image capturing protocol display area 150, the burden of selecting a protocol is reduced.

The control apparatus 100 transmits image capturing information common to the radiation detectors (first and second radiation detectors) 102 and 103 to the radiation detectors (first and second radiation detectors) 102 and 103.

The control apparatus 100 can list protocols by distinguishing between a group of protocols that can be commonly used by the radiation detectors 102 and 103, a group of protocols that can be used by the radiation detector 102, and a group of protocols that can be used by the radiation detector 103. Thus, according to the image capturing environment of radiation detectors (the number of radiation detectors and the features of radiation detectors), the operator can select a group of protocols to be displayed in the image capturing protocol display area 150.

That is, the operator can select a group of protocols from the group of protocols that can be commonly used by the radiation detectors 102 and 103, the group of protocols that can be used by the radiation detector 102, and the group of protocols that can be used by the radiation detector 103, and select a protocol from the selected group of protocols. Since a group of protocols is displayed in the image capturing protocol display area 150 according to the image capturing environment of radiation detectors, the burden of selecting a protocol is reduced.

If an image capturing protocol is settled using the protocol ID selection unit 507, the settled protocol ID is transmitted from a protocol ID transmission unit 506 to the control apparatus 100. In the control apparatus 100, a protocol ID reception unit 526 receives the protocol ID, and an image capturing protocol settlement unit 527 extracts a corresponding ID from an image capturing protocol database (DB) 524. Then, various parameters such as image processing parameters, a part, an image capturing direction, a patient direction, or annotation supplementary information are settled.

As described above, in a case where the detector A 102 is enabled by the enabling button (enabling unit) 107, image capturing information set by the image capturing protocol ID input unit (setting unit) 110 is transmitted to the control apparatus 100, which controls the detector A 102 and the detector B 103. In a case where the detector A 102 is enabled by the enabling button 107, the control apparatus 100 controls the detector A 102 and the detector B 103 based on image capturing information set by the image capturing protocol ID input unit 110.

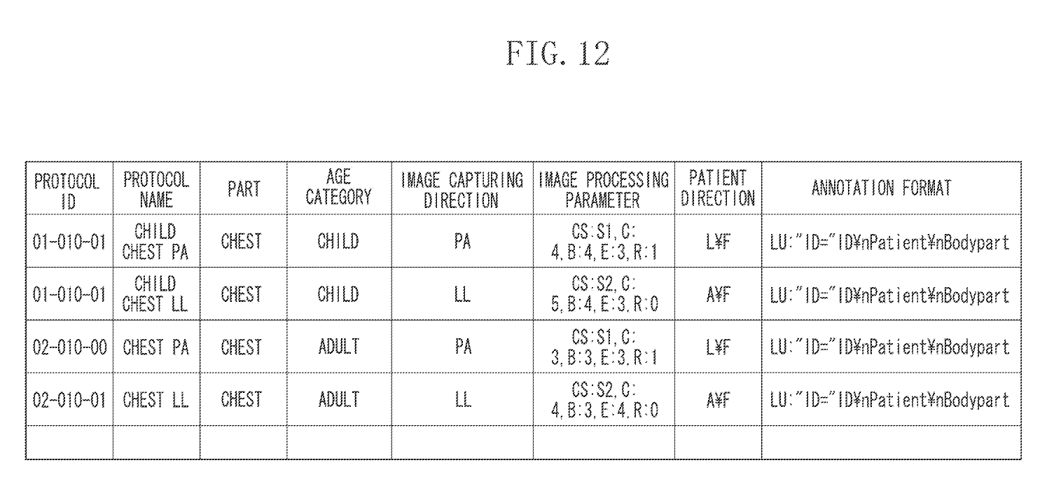

FIG. 12 illustrates an example of the image capturing protocol DB 524. The settled parameters are saved in an image capturing parameter information holding unit 528. Conventionally, to change protocols, the radiologic technologist needs to return from the imaging room to the operation room where the control apparatus 100 is set, and select a protocol again using a protocol ID transmission unit 542. In the present exemplary embodiment, however, in each radiation detector, instructions are given to enable and disable the radiation detectors and also specify an image capturing protocol.

After the radiologic technologist positions the patient's hand on the detector A 102 so that the wrist can be captured, and if the radiologic technologist presses the emission switch of the X-ray generating apparatus, X-rays are emitted, and a transmission image of the patient's wrist is read by an image data reading unit 508 of the detector A 102. The emission conditions at this time are transferred from the generating apparatus to the control apparatus 100 via a network (not illustrated) and recorded in a memory 535 for storing implementation information of the control apparatus 100. The implementation information includes a tube current, a tube voltage, an emission time, and grid information.

The read data is transferred from an image data transmission unit 509 to the control apparatus 100. In the control apparatus 100, an image data reception unit 530 receives the image data, and a temporary image holding unit 531 stores the image. The control apparatus 100 also notifies a control unit of the control apparatus 100 that the image data has been received. The control unit of the control apparatus 100 instructs an image processing unit 529 to perform various processes such as the density processing, the contrast processing, degree-of-emphasis processing, and noise reduction processing on the acquired image data, using the various image processing parameters set for the image capturing protocol.

An image rotation/vertical and horizontal inversion processing unit 532 performs predetermined rotation processing and predetermined inversion processing on the captured image subjected to image processing. A supplementary information addition unit 533 acquires patient information and implementation information from the patient information holding unit 534 and the implementation information holding unit 535 and attaches the patient information and the implementation information as header information to the image, thereby linking the patient with the image.

An annotation addition unit 536 displays the patient information and the implementation information at predetermined positions in the image according to an annotation format. The thus generated quality assurance (QA) image is stored in a captured image storage unit 537, cropped to a predetermined size, and then output to a picture archiving and communication system (PACS) or a printer.

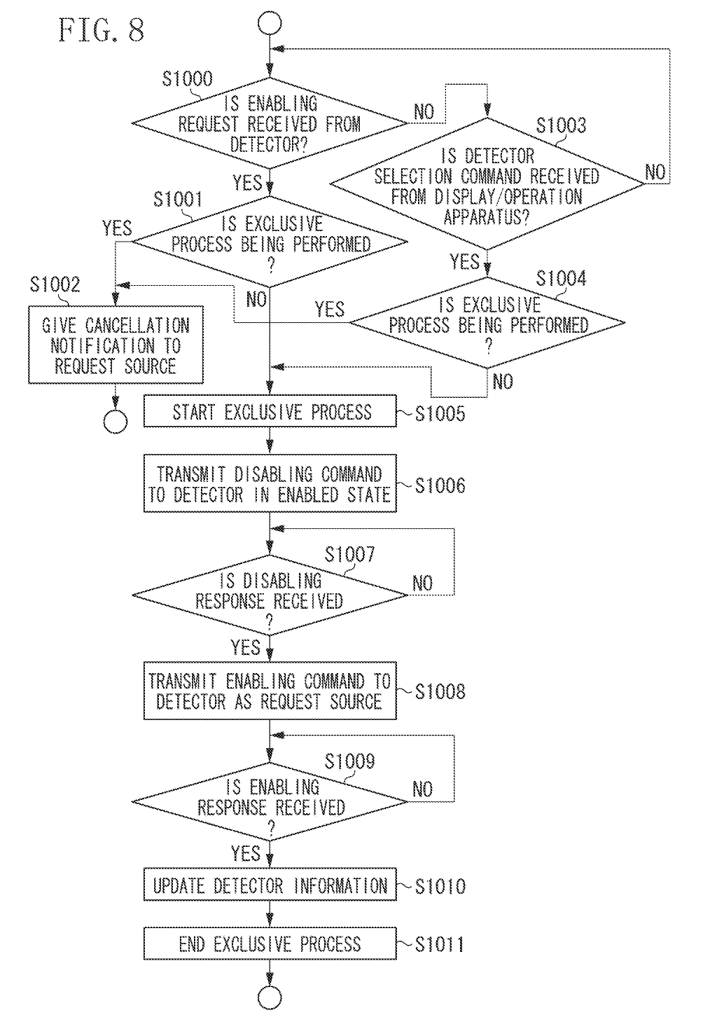

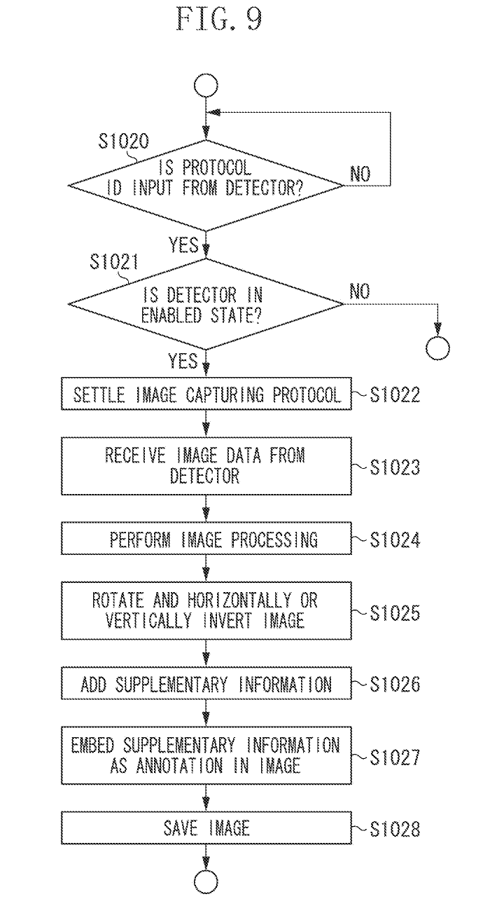

FIG. 8 is a flowchart in a case where a radiation detector is enabled. FIG. 9 is a flowchart in a case where a protocol is selected. The flow of processing in the present exemplary embodiment will be described with reference to FIGS. 8 and 9.

As illustrated in FIG. 8, if the radiologic technologist presses the enabling button 107 of the detector A 102 that the radiologic technologist wishes to enable, the detector A 102 transmits an enabling request to the control apparatus 100. In step S1000, if the control apparatus 100 receives an enabling request from the detector A 102 (YES in step S1000), then in step S1001, the control apparatus 100 confirms whether an exclusive process is being performed. If an exclusive process is being performed (YES in step S1001), then in step S1002, the control apparatus 100 provides a cancellation notification to the detector A 102 as the request source, thereby notifying the detector A 102 that the enabling request has not been accepted.

If an exclusive process is not being performed (NO in step S1001), then in step S1005, the control apparatus 100 starts an exclusive process. From this point onward, the control apparatus 100 will not accept another enabling request or a disabling request until the exclusive process is completed. Next, in step S1006, the control apparatus 100 transmits a disabling command to the detector B 103 in the enabled state. If the detector B 103 is disabled and transitions to the disabled state, the detector B 103 transmits a disabling response.

In step S1007, if the control apparatus 100 receives the disabling response (YES in step S1007), then in step S1008, the control apparatus 100 transmits an enabling command to the detector A 102 as the request source. If the detector A 102 is enabled and transitions to the enabled state, the detector A 102 transmits an enabling response. In step S1009, if the control apparatus 100 receives the enabling response (YES in step S1009), the control apparatus 100 updates detector information in the detector information holding unit 522. In step S1011, the control apparatus 100 ends the exclusive process.

If the control apparatus 100 does not receive an enabling request from the detector A 102 in step S1000 (NO in step S1000), then in step S1003, the control apparatus 100 confirms whether the control apparatus 100 receives a detector selection command from the display/operation apparatus 101. If the control apparatus 100 does not receive a detector selection command (NO in step S1003), the processing returns to step S1000. If the control apparatus 100 receives a detector selection command (YES in step S1003), then in step S1004, the control apparatus 100 confirms whether an exclusive process is being performed. If an exclusive process is being performed (YES in step S1004), then in step S1002, the control apparatus 100 provides a cancellation notification to the display/operation apparatus 101. If an exclusive process is not being performed (NO in step S1004), the processing proceeds to step S1005 and thereafter, and the radiation detectors are switched.

In FIG. 9, the radiologic technologist selects a protocol ID using the protocol ID selection unit 507 on a radiation detector. As a result, the protocol ID is sent to the control apparatus 100. Then, in step S1020, the control apparatus 100 confirms whether a protocol ID is input from a radiation detector. If a protocol ID is input (YES in step S1020), then in step S1021, the control apparatus 100 confirms whether the radiation detector is in the enabled state. If the radiation detector is in the enabled state (YES in step S1021), then in step S1022, the control apparatus 100 settles an image capturing protocol. If the radiation detector is not in the enabled state (NO in step S1021), this means that the protocol ID has been sent at a timing when the protocol ID should not be sent. Thus, a message to that effect is displayed, and this operation is cancelled.

Next, after an image is captured, in step S1023, the control apparatus 100 acquires image data from the radiation detector and stores the image data in the temporary image holding unit 531. In step S1024, the control apparatus 100 performs image processing using image processing parameters set for each protocol. In step S1025, based on the instructions of rotation and inversion set for each protocol, the control apparatus 100 performs desired rotation processing and desired inversion processing.

In step S1026, the control apparatus 100 acquires patient information and implementation information from the patient information holding unit 534 and the implementation information holding unit 535, respectively, and adds the patient information and the implementation information as header information to the image data. The control apparatus 100 displays the image on a screen according to an annotation format. In step S1028, the control apparatus 100 stores the thus generated image in the captured image storage unit 537.

Conventionally, to select another radiation detector or another protocol ID while positioning the patient in the imaging room, the radiologic technologist needs to return to the operation room and perform an operation using the display/operation apparatus 101 of the control apparatus 100. In the present exemplary embodiment, however, the enabling instruction unit 500 and the protocol ID selection unit 507 attached to a radiation detector are selected, whereby an image can be captured. In the above example, providing an enabling instruction and the selection of a protocol are separately performed. Alternatively, a protocol ID can be selected, thereby simultaneously transferring an enabling instruction and the image capturing protocol ID.

The protocol ID list illustrated in FIG. 3 differs for each radiation detector and is a list of protocol IDs with which an image can be captured by a corresponding radiation detector. The order of protocol IDs in the list can be automatically rearranged in the order of use frequency or can be customized by a user. Each radiation detector and the control apparatus 100 can perform wired communication with each other or can perform wireless communication with each other.

As each radiation detector, a radiation detector of a type provided with a memory for storing a captured image is applicable. In this case, a unit for storing image capturing information input from an image capturing information selection unit provided on the radiation detector side, in association with a captured image, is provided, whereby various processes can be executed based on an image capturing protocol on the control apparatus 100 side.

That is, when a captured image stored in the radiation detector after an examination is sent to the control apparatus 100, image capturing information settled when the image had been captured can be sent together with the image. Thus, it is possible to perform image processing suitable for an image capturing technique without specifying an image capturing protocol on the control apparatus 100 side.

As described above, according to the present exemplary embodiment, a radiation detector is enabled by an enabling unit provided in a housing or a support for accommodating the radiation detector, and image capturing information is set by a setting unit also provided in the housing or the support, whereby the radiation detector can be controlled at the installation location of the radiation detector.

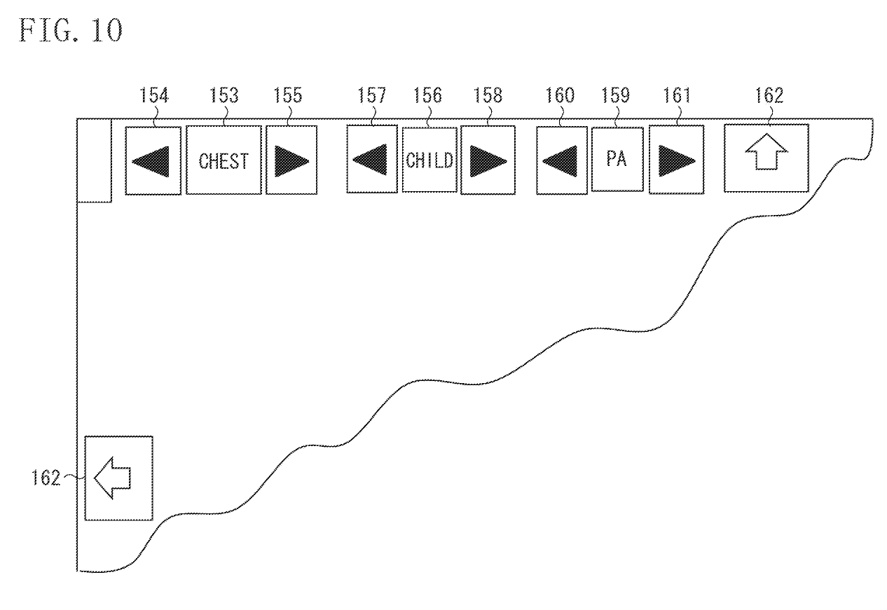

A second exemplary embodiment will be described with reference to FIGS. 10 to 14. Components, functions, and operations similar to those in the above exemplary embodiment will not be described, and the differences from the above exemplary embodiment will mainly be described.

In the first exemplary embodiment, an image capturing protocol ID is input through the image capturing protocol ID input unit 110 and transmitted to the control apparatus 100, whereby a corresponding image capturing technique can be performed. In the present exemplary embodiment, an example is illustrated where a part, an age category, an image capturing direction, and rotation information are input.

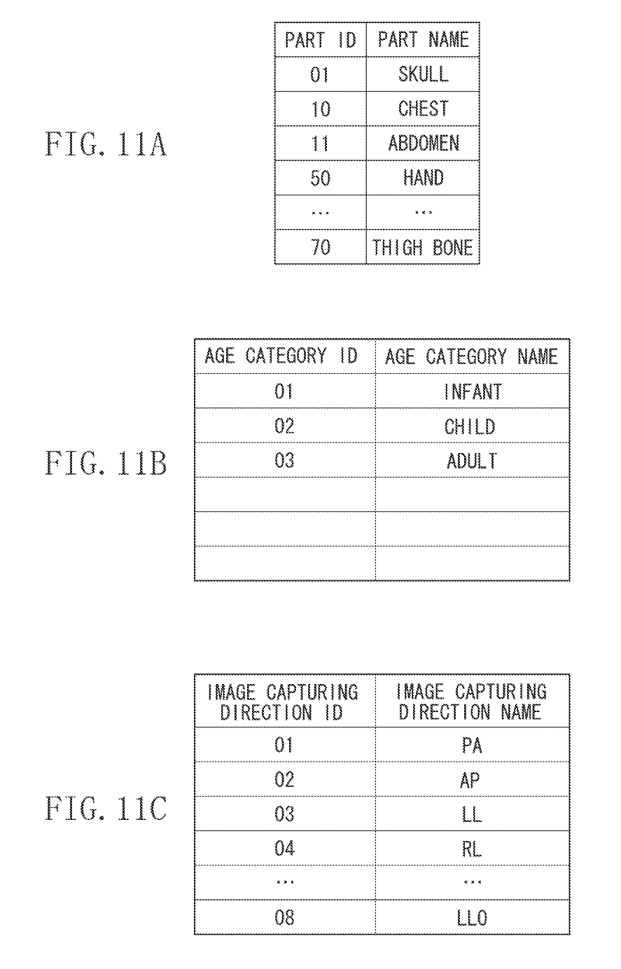

FIG. 10 illustrates input units for inputting a part, an age category, an image capturing direction, and rotation information. FIG. 10 illustrates a part display area 153, which displays a part, an age category display area 156, which displays an age category, and an image capturing direction display area 159, which displays an image capturing direction. The display item of the part display area 153 can be changed using change buttons 154 and 155. The display item of the age category display area 156 can be changed using change buttons 157 and 158. The display item of the image capturing direction display area 159 can be changed using change buttons 160 and 161. FIGS. 11A to 11C illustrate lists of parts (FIG. 11A), age categories (FIG. 11B), and image capturing directions (FIG. 11C). The items to be displayed are selected from these lists.

FIG. 12 illustrates the relationships among a protocol ID, a part, an age category, and an image capturing direction. If a part, an age category, and an image capturing direction are input and determined, a protocol ID is determined, and image processing parameters, a patient direction value, and an annotation format are determined.

Top indication buttons (a direction specifying unit) 162 are provided in a housing or a support for accommodating each of the detectors 102 and 103, and a direction coinciding with the direction of a displayed radiographic image is specified using the top indication buttons 162. The top indication buttons 162 are attached to upper, lower, left, and right portions of each of the detectors 102 and 103. One of the top indication buttons 162 indicating a direction coinciding with the up direction of a displayed image is specified, whereby rotation information regarding the rotation of the image from a data reading direction can be conveyed.

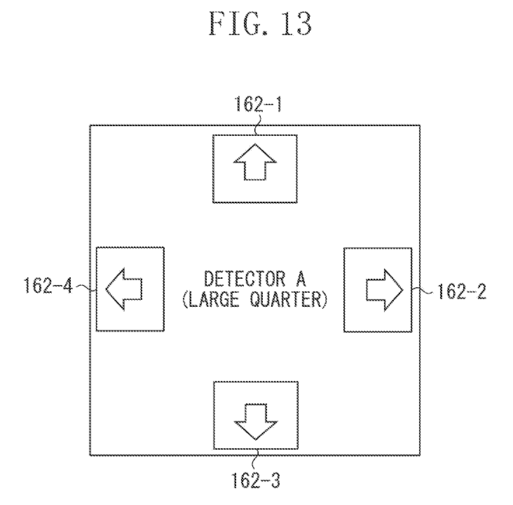

That is, as illustrated in FIG. 13, the top indication buttons 162 corresponding to the up direction of a displayed image are provided in four directions of each of the detectors 102 and 103. Then, one of the top indication buttons 162 indicating a direction coinciding with the up direction of a displayed image is pressed, thereby specifying the up direction of the displayed image.

For example, the direction of a top indication button 162-1 is set in advance to an up direction when an image acquired in a data reading direction is displayed. In this case, if each of top indication buttons 162-1, 162-2, 162-3, and 162-4 is specified, the up direction of the displayed image rotates 0°, 90°, 180°, and 270° clockwise with respect to the up direction set in advance. Thus, the image read in the data reading direction is rotated 0°, 90°, 180°, and 270° counterclockwise according to the direction of a pressed top indication button 162, whereby it is possible to display the image in a correct direction.

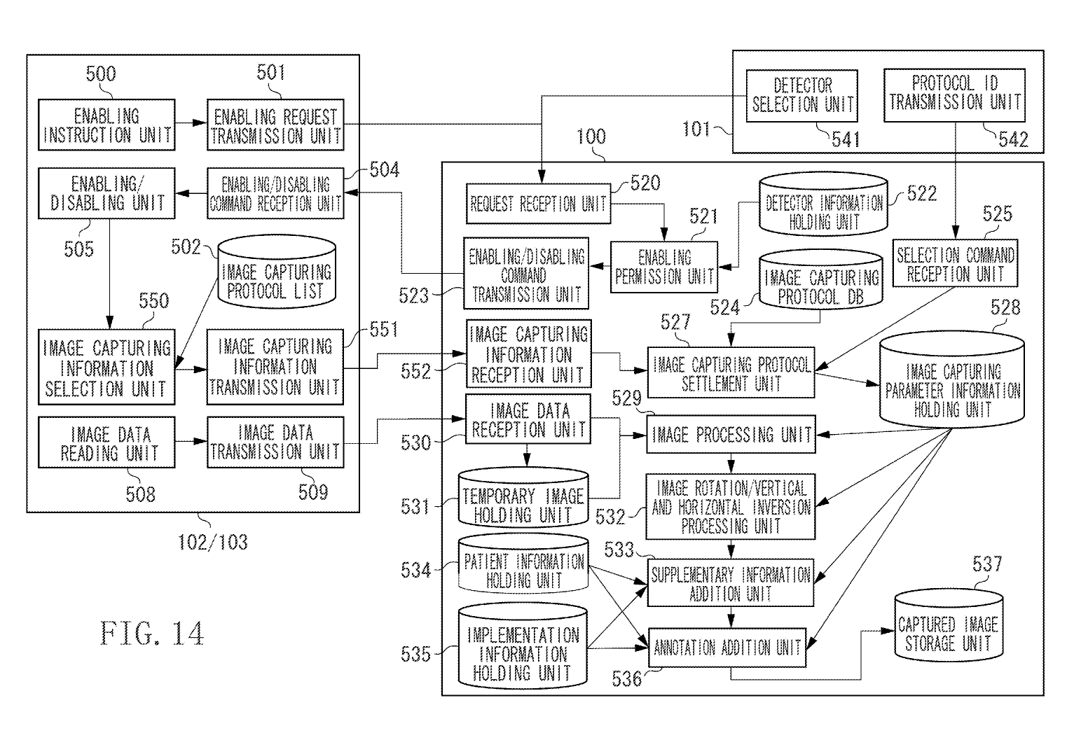

FIG. 14 is a block diagram illustrating an example of a radiographic system according to the present exemplary embodiment. Components similar to those in FIG. 7 are designated by the same numerals.

An image capturing information selection unit 550, which is included in each of the detectors 102 and 103, selects a part, an age category, an image capturing direction, and a top indication according to input from the change buttons in FIG. 10. Based on the part, the age category, and the image capturing direction selected by the image capturing information selection unit 550, a protocol ID is determined from the table in FIG. 12.

One of the top indication buttons 162 is pressed, whereby a rotation angle with respect to the up direction set in advance is recognized as rotation information. As image capturing information, the protocol ID and the rotation information are sent from an image capturing information transmission unit 551 to an image capturing information reception unit 552 of the control apparatus 100. Based on the protocol ID sent to the image capturing information reception unit 552, the image capturing protocol settlement unit 527 settles an image capturing protocol and saves preset image capturing parameters in the memory 528. Based on the rotation information sent to the image capturing information reception unit 552, parameters for rotation saved in the memory 528 are updated. The rest of the processing is similar to that in the first exemplary embodiment.

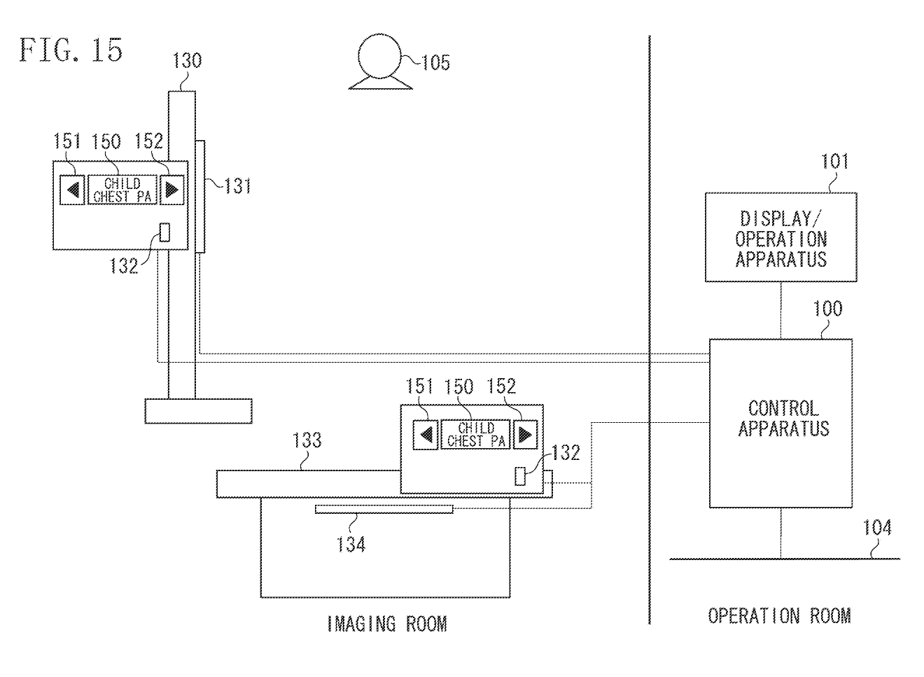

FIG. 15 illustrates a schematic diagram of a system according to a third exemplary embodiment. Components, functions, and operations similar to those in the above exemplary embodiments will not be described, and the differences from the above exemplary embodiments will mainly be described.

In the first and second exemplary embodiments, cassette-type radiation detectors are used. Thus, operation units such as the enabling button 107, the disabling button 108, and the image capturing information input unit (the image capturing protocol ID input unit 110) are provided on each of the detectors 102 and 103. In the present exemplary embodiment, in a method where cassette-type radiation detectors are attached to a support, it is easy to operate these operation units, regardless of the attachment position of each of the detectors 102 and 103.

For example, the enabling button 107, the disabling button 108, and the image capturing information input unit (the image capturing protocol ID input unit 110) can be provided on a support to which each radiation detector is attached.

An enabling/disabling button 132 and image capturing protocol input units 150 to 152, which are examples of image capturing information input units, in FIG. 15 can be provided on each of a stand 130, which is a standing-type support, and a bed 133, which is a bed-type support.

In the above exemplary embodiments, after another radiation detector (the detector B 103) is disabled, a radiation detector (the detector A 102) is enabled. In another exemplary embodiment, based on an enabling request from a radiation detector (the detector A 102), the process of enabling another radiation detector (the detector B 103) associated with the radiation detector and in the disabled state can be performed. That is, in a case where the detector A 102 is enabled, the enabling button (enabling unit) 107 enables the detector B 103.

In this case, image capturing information set by the image capturing protocol ID input unit (setting unit) 110 can be transmitted to the detector B 103, and the same image capturing information as that of the detector A 102 can be set for the detector B 103.

In a case where a memory for storing an acquired image is provided in an FPD (a radiation detector), the radiation detector can include a memory (a holding unit) for storing a radiographic image generated based on detected radiation, in association with image capturing information.

### Other Embodiments

Embodiments can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While exemplary embodiments have been provided, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-144772, filed Jul. 22, 2016, which is hereby incorporated by reference herein in its entirety.

What is claimed is:

1. A radiographic apparatus comprising:
an enabling unit provided in a first radiation detector and configured to enable the first radiation detector;
a setting unit provided in the first radiation detector and configured to set image capturing information of the first radiation detector; and
a control unit configured to, in a case where the first radiation detector is enabled by the enabling unit, control the first radiation detector and a second radiation detector based on the image capturing information set by the setting unit.

2. The radiographic apparatus according to claim 1, wherein the first radiation detector includes a holding unit configured to store a radiographic image generated based on detected radiation in association with the image capturing information.

3. The radiographic apparatus according to claim 1, wherein the image capturing information includes at least one of an image capturing protocol identification (ID), a captured part, an image capturing direction, an age category of a subject, and rotation information of a radiographic image.

4. The radiographic apparatus according to claim 1, wherein the setting unit sets the image capturing information of the first radiation detector from the image capturing information ordered according to priority based on past image capturing information of at least one of the first radiation detector and the second radiation detector.

5. The radiographic apparatus according to claim 4, wherein the priority is determined based on a setting frequency or a setting time of the image capturing information.

6. The radiographic apparatus according to claim 1, wherein the control unit transmits the image capturing information common to the first radiation detector and the second radiation detector to the first radiation detector.

7. The radiographic apparatus according to claim 1, further comprising a direction specifying unit provided in the first radiation detector and configured to specify a direction coinciding with a direction of a displayed radiographic image.

8. The radiographic apparatus according to claim 1, wherein in a case where the enabling unit enables the first radiation detector, the enabling unit enables the second radiation detector.

9. The radiographic apparatus according to claim 8, wherein the image capturing information set by the setting unit is transmitted to the second radiation detector.

10. A radiographic apparatus comprising:
an enabling unit provided in a housing for accommodating a first radiation detection unit for detecting radiation and configured to enable the first radiation detection unit; and
a setting unit provided in the housing and configured to set image capturing information of the first radiation detection unit,
wherein in a case where the first radiation detection unit is enabled by the enabling unit, the image capturing information set by the setting unit is transmitted to a control unit configured to control the first radiation detection unit and a second radiation detection unit.

11. A radiographic apparatus comprising:
an enabling unit provided in a support for accommodating a first radiation detection unit for detecting radiation and configured to enable the first radiation detection unit; and
a setting unit provided in the support and configured to set image capturing information of the first radiation detection unit,
wherein in a case where the first radiation detection unit is enabled by the enabling unit, the image capturing information set by the setting unit is transmitted to a control unit configured to control the first radiation detection unit and a second radiation detection unit.

12. A radiographic apparatus comprising:
an enabling unit provided in a radiation detector and configured to enable the radiation detector;
a setting unit provided in the radiation detector and configured to set image capturing information of the radiation detector; and
a control unit configured to, in a case where the radiation detector is enabled by the enabling unit, control the radiation detector based on the image capturing information set by the setting unit.

13. A radiographic system comprising:
a radiation generation unit configured to generate radiation;
a first radiation detector configured to detect the generated radiation;
a second radiation detector configured to detect the generated radiation; and
a control unit configured to control the radiation generation unit, the first radiation detector, and the second radiation detector,
wherein the first radiation detector includes:
an enabling unit configured to enable the first radiation detector; and
a setting unit configured to set image capturing information of the first radiation detector,
wherein in a case where the first radiation detector is enabled by the enabling unit, the control unit controls the first radiation detector and the second radiation detector based on the image capturing information set by the setting unit.

14. A radiographic method, comprising:
enabling a first radiation detector;
setting image capturing information of the first radiation detector; and
controlling, in a case where the first radiation detector is enabled, based on image capturing information of the first radiation detector, the first radiation detector and a second radiation detector.

15. A computer-readable storage medium storing computer-executable instructions for causing a computer to execute a radiographic method, the radiographic method comprising:
enabling a first radiation detector;
setting image capturing information of the first radiation detector; and
controlling, in a case where the first radiation detector is enabled, based on image capturing information of the first radiation detector, the first radiation detector and a second radiation detector.
